(19) United States
(12) Patent Application Publication
(10) Pub. No.: US 2006/0025712 A1
(43) Pub. Date: Feb. 2, 2006

(54) Donning facilitator for dorsal wrap-around ankle-foot orthosis

(76) Inventor: Donald James Kammerer, Brentwood, CA (US)

Correspondence Address: Donald J. Kammerer, 437 Apple Hill Dr., Brentwood, CA, 94513, US

(21) Appl. No.: 10/900,583

(22) Filed: Jul. 27, 2004

(51) Int. Cl. A61F 13/00; A61F 13/06

(52) U.S. Cl. 602/65; 602/60

(57) ABSTRACT

A hand held adaptive device that allows a disabled person an effective and easy way to don their dorsal wrap-around DAFO (Dynamic Ankle Foot Orthosis)(38). The Donning Facilitator (40) slides in-between the dorsal wrap-around supports (39), thus separating and maintaining an enlarged opening of the anterior-dorsal aspect of the dorsal wrap-around DAFO (38). The impaired foot slides easily into the enlarged opening of the said dorsal wrap-around DAFO (38). In addition, caregivers will find the Donning Facilitator (40) frees up both hands from having to hold open the bilateral dorsal wrap-around supports (39), allowing ample control by the caregiver when donning the dorsal wrap-around DAFO (38) to the patient's foot/ankle.

### CROSS-REFERENCE TO RELATED APPLICATIONS

None.

### FEDERALLY SPONSORED RESEARCH

None.

### SEQUENCE LISTING

Not Applicable.

### BACKGROUND OF THE INVENTION-FIELD OF INVENTION

This invention relates to disabled person's ability to independently don a dorsal wrap-around dynamic ankle foot orthotic (herinafter a DAFO), specifically an adaptive device that opens the dorsal wrap around portion of the dorsal wrap-around DAFO so the impaired foot can slide down into the created enlarged opening with minimal effort on the part of the disabled person or caregiver.

### BACKGROUND OF THE INVENTION

A dorsal wrap-around (dynamic) Ankle Foot Orthosis (herinafter a DAFO) is a medical device that provides support to the patient's ankle and foot. The Donning Facilitator will allow a disabled person (child or adult) the opportunity to put on their dorsal wrap-around DAFO at a modified independent level. In addition the Donning Facilitator provides a caregiver increased ease and assurance to not startle or hurt the patient when donning the dorsal wrap-around DAFO. In this case the benefit is specifically for the caregiver because without the Donning Facilitator it takes two hands to open the dorsal wrap-around portion of the DAFO and a third to lift and place the patient's foot into the dorsal wrap-around DAFO. If the dorsal wrap-around DAFO slips out of the caregiver's hands while holding the bilateral dorsal wrap-around supports open it will snap closed, startle and hurt the patient. Then it is likely that the patient will develop an aversion to the placement of the dorsal wrap-around DAFO and result in greater hyper tonicity, thus making it much more difficult to don the dorsal wrap-around DAFO.

The general problem is that when a disabled person requires the use of a dorsal wrap-around DAFO there is significant difficulty with putting it on the disabled person's foot. The needed ankle support that is provided by the dorsal wrap-around DAFO is seen in the rigidity through out the dorsal wrap-around DAFO. The bilateral dorsal wrap-around supports is often too difficult to open even when both hands are functionally available to use. If the patient can open the bilateral dorsal wrap-around supports with both hands, he or she still have to reach down to the affected foot to don the dorsal wrap-around DAFO. This requires a high level of strength, balance and coordination. The overall process is often impossible for an impaired person to accomplish.

The limiting factor of donning the dorsal wrap-around DAFO lies with the disabled person's associated deficits that impair the individual's functional ability. The common deficits are seen in the diagnosis of cerebral palsy; hemiplegia (paralysis of one side of the body), spastic diplegia (paralysis of corresponding parts on both sides of the body), spastic quadriplegia (paralysis of all four limbs), hyper tonicity (having a greater degree of tension in the muscles), hypo tonicity (having a lesser degree of tension in the muscles), cerebral vascular accident, arthrogryposis, but not limited to just these diagnosis. The associated deficits commonly seen are poor trunk balance or instability, generalized weakness, incoordination and or paralysis of one or more extremities. This results in most cases the need to use at least one upper extremity for support or just simply having only one upper extremity to utilize with donning the dorsal wrap-around DAFO. What commonly happens is the disabled person is unable to don the dorsal wrap-around DAFO by them selves and becomes dependent on others to don the dorsal wrap-around DAFO.

The problem of becoming dependent on a caregiver or family member to don the dorsal wrap-around DAFO on a daily basis can lead to limited self-confidence and frustration on the part of the disabled person. Then the issue of actually using the dorsal wrap-around DAFO becomes a greater problem and the parents or caregivers become frustrated and everyone involved begins to minimize the importance of such a brace and foregoes wearing it. This can result in the disabled persons increased risk for falls; the ankle joint can be in jeopardy of a fracture and or associated injuries. The neuro-motor pattern or positioning of the ankle can result in a muscle contracture at the joint. Ultimately, the disabled person jeopardizes his or her own ability to weight bear through the impaired lower extremity. The ability to transfer, stand and ambulate becomes too difficult, too painful and or a high risk for a fall.

The majority of people that require the dorsal wrap-around DAFO have poor dorsi-flexion, poor plantar-flexion at the ankle and poor peak knee flexion during swing/stance phase. Without the dorsal wrap-around DAFO the ankle will result in “foot drop”. The necessity of the dorsal wrap-around DAFO is to lift the foot into peak dorsi-flexion during gait and not drag or catch their toes on the ground. Another purpose of the dorsal wrap-around DAFO is to maintain proper joint alignment and prevent contractures from occurring at the ankle and foot.

My experience and the research I have read shows that the dorsal wrap-around DAFO is the ankle foot orthosis of choice with physicians and therapists because of the greater control it provides with the degree of dynamic dorsi-flexion and plantar-flexion permitted at the ankle/foot. It has been documented in the Journal “BIOMECHANICS”, August 2003 p. 13, 14 Titled: Research fails to settle debate over AFO's for spastic diplegia. It was identified “One thing parents say to me is that children can put on the articulating (hinged) AFO by themselves,” said miller “But they cannot put on the wrap-around (dynamic) AFO by them selves, so becomes an issue of dependency.”—Jordana Bier.

This has been my finding also: the majority of patients that use the dorsal wrap-around DAFO cannot put them on by themselves, so it becomes an issue of dependency. The only other option is to change the dorsal wrap-around DAFO to a (hinged) Ankle Foot Orthosis (herinafter an AFO). The hinged AFO doesn't have the dorsal wrap-around supports and provides less than the optimal support desired, therefore it is less beneficial than the dorsal wrap-around DAFO. However most disabled people can with little difficulty don the hinged AFO. Therefore, staying with the dorsal wrap-around DAFO the patient gives up his or her functional independence that is related to donning the dorsal wrap-around DAFO.

I have invented the Donning Facilitator for the dorsal wrap-around (dynamic) Ankle Foot Orthosis (DAFO) and my testing has shown that with the Donning Facilitator patients are successful in donning their own dorsal wrap-around DAFO. I believe the Donning Facilitator will maximize a disabled person's functional independence and that person's quality of life will improve.

### BACKGROUND-DISCUSSION OF PRIOR ART

My patent search revealed various devices that relate to the donning of foot wear such as socks, shoes and one that assists with donning a generic AFO without the dorsal wrap-around supports of a (dynamic) Ankle Foot Orthosis (DAFO), however none offer the necessary solution to the identified problem stated above.

U.S. Pat. No. 6,409,692 B1 to Ann Marie Covey describes an Ankle Foot Orthosis (AFO) donning device used in aiding a person in donning an Ankle Foot Orthosis (AFO) and their shoe at the same time. This device holds the patient's shoe with the AFO already placed inside the shoe, then the patient slides the foot into the shoe with the assist of the guide. This device only works for the “standard” or generic type of AFO, which does not have the component of the dorsal wrap-around supports located on the anterior-dorsal aspect of the dorsal wrap-around DAFO. This device does not work for the dorsal wrap-around DAFO because there is no solution to separate and hold open the dorsal wrap-around supports necessary for the patient's foot to slide into position.

U.S. Pat. App. No. 2002/0139819 A1 to Michael P. Ferraioli describes a footwear-donning device designed for the removal, retrieval and/or donning of footwear, footwear such as a sock, shoe and other footwear. The device comprises of a carriage for holding the footwear and an extended handle. This device does not solve the problem of donning the dorsal wrap-around DAFO by separating and holding open the dorsal wrap-around supports of the DAFO necessary for the patient's foot to slide into position. This device does not meet the needs for donning any type of Ankle foot Orthosis (AFO).

U.S. Pat. No. 4,943,097 to Sampson D. Sanger describes a manually operable personal convenience implement consisting of a single longitudinal arm with a wedged half conical, designed to assist in donning and removing shoes, stockings, hose, underwear and trousers. The device was not intended and does not solve the problem of donning a dorsal wrap-around DAFO. The device cannot be used to separate and hold open the dorsal wrap-around supports of a dorsal wrap-around DAFO necessary for the patient's foot to slide into position. This device does not meet the needs for donning any type of Ankle foot Orthosis (AFO).

U.S. Pat. No. 6,283,342 to Melvin T. Wilkerson describes a footwear donning assistive device. The device comprises of a handle and an insertion member that which guides the user's heel into the shoe preventing portions of the shoe from being folded or rolled when being donned. This device was not intended and does not solve the problem of donning a dorsal wrap-around DAFO. The device cannot be used to separate and hold open the dorsal wrap-around supports of a dorsal wrap-around DAFO necessary for the patient's foot to slide into position. This device does not meet the needs for donning any type of Ankle foot Orthosis (AFO).

U.S. Pat. No. 5,687,889 to Douglas T. Liden describes a multi-purpose reacher and dressing aid. A manual device designed to assist with donning and doffing socks, don shoes, pick up articles from the floor, tighten shoelaces and donning pants. This device was not intended and does not solve the problem of donning a dorsal wrap-around DAFO. The device cannot be used to separate and hold open the dorsal wrap-around supports of a dorsal wrap-around DAFO necessary for the patient's foot to slide into position. This device does not meet the needs for donning any type of Ankle foot Orthosis (AFO).

U.S. Pat. No. 5,974,701 to Virginia G. Busch describes a shoe donning enabler designed to be used with or without a dressing stick. This device is intended to assist with donning a shoe only. This device was not intended and does not solve the problem of donning a dorsal wrap-around DAFO. The device cannot be used to separate and hold open the dorsal wrap-around supports of a dorsal wrap-around DAFO necessary for the patient's foot to slide into position. This device does not meet the needs for donning any type of Ankle foot Orthosis (AFO).

U.S. Pat. No. 4,683,876 to Valley Changras describes an orthopedic guide that serves as an aid to an individual having a leg injury requiring a leg brace. The device stabilizes both shoe and leg brace placed there in and aids in putting on a leg brace and shoe at the same time. This device cannot be used to separate and hold open the dorsal wrap-around supports of the dorsal wrap-around DAFO necessary for the patient's foot to slide into position. Therefore this device does not meet the needs for donning the dorsal wrap-around DAFO.

These devices fall short of identifying and solving the problem of donning the dorsal wrap-around DAFO necessary for the patient's foot to slide into position. Therefore a clear and unsolved need for a hand held assistive device that minimizes the struggle with donning the dorsal wrap-around DAFO. The Donning Facilitator removes the most common and difficult component associated in donning the dorsal wrap-around DAFO. The Donning Facilitator separates and holds the bilateral dorsal wrap-around supports in a created enlarged opening of the anterior-dorsal aspect of the DAFO allowing the impaired foot to slide into position relatively easily.

### BACKGROUND OF INVENTION-OBJECTS AND ADVANTAGES

Accordingly, several objects and advantages of my invention are that the Donning Facilitator removes the difficulty of donning the dorsal wrap-around DAFO, providing modified independence to the patient and significant ease on the part of the caregiver:
(a) To provide the disabled person a tool to use in separating the bilateral dorsal wrap-around supports of the DAFO into an enlarged opening allowing the impaired foot to slide into its desired position.
(b) To provide the disabled person the minimum amount of effort necessary to don the dorsal wrap-around DAFO and to maximize the level of success.
(c) To provide greater compliance of wearing the dorsal wrap-around DAFO by creating an improved self-confidence and gratification resulting by the improved ease of donning the dorsal wrap-around DAFO.
(d) To provide the disabled person the ability to don the dorsal wrap-around DAFO independently and avoid the frustration commonly associated with donning the dorsal wrap-around DAFO.
(e) To provide the disabled person an improved method of donning their dorsal wrap-around DAFO by sliding their foot down into the created enlarged opening. The method makes it possible for a person to slide the Donning Facilitator in to position on the dorsal wrap-around DAFO. Then don the dorsal wrap-around DAFO to the impaired ankle and foot, requiring the use of only one hand, similar to putting on an open boot with the use of one hand.
(f) The Donning Facilitator will slide backwards and out from between the dorsal wrap-around supports as the foot descends down into the created enlarged opening of the dorsal wrap-around DAFO allowing ample space for the foot to slide into the proper position.
(g) To provide the patient such as a child with cerebral palsy or an adult with hemiplegia, both commonly present with impaired upper and lower extremities. The impairments can affect a person in a combination of ways such as the right upper and lower extremity lacking functional use. Therefore the Donning Facilitator provides the ability to accomplish the task of donning the dorsal wrap-around DAFO in a modified independent way utilizing the one functional upper extremity.
(h) To provide the caregiver or parent increased ease and assurance to not startle or hurt the patient when donning the dorsal wrap-around DAFO. The dorsal wrap-around support is designed to custom form fit the top portion of the impaired foot and ankle. When they are separated apart there is resistance or pressure so that if the bilateral dorsal wrap-around supports slip out of the caregivers hands while attempting to put the patient's foot into the dorsal wrap-around DAFO this would result in a snap response and would likely startle and hurt the patient's foot. The patient may develop an aversion to the placement of the dorsal wrap-around DAFO. If this were to occur, then greater hyper tonicity would result and would cause greater difficulty with donning the dorsal wrap-around DAFO.
(i) To provide the caregiver decreased anxiety with the improved ease to manipulate the patients impaired foot into the created enlarged opening of the dorsal wrap-around DAFO, by already having the Donning Facilitator in place and the dorsal wrap-around supports fully open. With out the use of the Donning Facilitator this process requires two hands to safely open each side of the wrap-around supports and hold the dorsal wrap-around DAFO in position, while having to then put the patient's foot into the dorsal wrap-around DAFO. This process is far more difficult when the patient is severely disabled and the foot and ankle requires prior stretching to the calf muscles and dependent assist to lift and position it into the dorsal wrap-around DAFO.

Further objects and advantages of my invention are that a disabled person now has a greater advantage in accomplishing the task of donning their dorsal wrap-around DAFO in a modified independent way. With the use of the Donning Facilitator patients, parents and caregivers alike will benefit. The patient won't have anxiety or frustration over having to put on their dorsal wrap-around DAFO on a daily basis. The parent or caregiver can rest assured they won't cause the patient pain when putting the patient's foot into the dorsal wrap-around DAFO. Other people interested in this problem being solved are the patient's occupational therapist, physical therapist, physician and orthotist. Everyone involved in the patient's well-being will appreciate the significant benefits the Donning Facilitator brings to donning a dorsal wrap-around DAFO. Still further objects and advantages will become apparent from a consideration of the drawings and ensuing description.

### SUMMARY

The Donning Facilitator is a hand held adaptive device which slides up between the bilateral dorsal wrap-around supports of the anterior-dorsal aspect of the of the dorsal wrap-around DAFO, separating and holding open the dorsal wrap-around supports to create an enlarged opening. This allows a disabled person the ability to don the dorsal wrap-around DAFO to their foot, or a caregiver can don the dorsal wrap-around DAFO with ease and without fear of causing pain to the patient's foot.

### DRAWINGS—FIGURES

In the drawings the Donning Facilitator is composed of individual components that are then molded to the desired angles and arches, finally adhered to each other with adhesive making the Donning Facilitator for dorsal wrap-around (dynamic) ankle-foot Orthosis complete.

FIG. 1a shows the side view relationship between the Donning Facilitator and the closed position of the dorsal wrap-around supports of the dorsal wrap-around DAFO.

FIG. 1b shows the side view of the Donning Facilitator separating the dorsal wrap-around supports of the dorsal wrap-around DAFO causing an enlarged opening.

FIG. 2a shows the front view of the anterior-dorsal aspect of the dorsal wrap-around supports of the dorsal wrap-around DAFO.

FIG. 2b shows the enlarged opening caused by the separation between the Donning Facilitator and the dorsal wrap-around supports of the dorsal wrap-around DAFO.

FIG. 3 shows top view, identifying the flared wings of the butterfly top, the dividers' anterior apex or point of reference and the relationship to the handle.

FIG. 4 shows the bottom-side view, identifying the bend between the handle and the dorsal arch base.

FIG. 5 shows the bottom view, identifying the relationship between the three individual pieces together, the dorsal arch base, the divider with its anterior apex and the four individual wings of the butterfly top.

FIG. 6 shows the bottom view of the Donning Facilitator and its bilateral grooves in which the dorsal wrap-around supports of the dorsal wrap-around DAFO slide into and then separate apart.

FIG. 7a shows the individual bottom component that includes the handle and dorsal arch base. Then molded to the desired angles and arches necessary to form the Donning Facilitator.

FIG. 7b shows the individual divider component with its anterior apex and posterior aspect.

FIG. 7c shows the individual butterfly top component with its bilateral greater and inferior wings, also the anterior and posterior notches.

FIG. 7d shows the individual fourth and optional component the clip and clip neck.

FIG. 8 shows the view of the Donning Facilitator from the bottom angle and its bilateral grooves in which the dorsal wrap-around supports of the dorsal wrap-around DAFO slide into and separate apart. The optional clip adheres to the back of the handle and separates prior to the bend. It remains centered and slightly spaced from the dorsal arch base.

FIG. 9 shows the bottom view, identifying the relationship between the four individual pieces together, including the distal end of the clip at the center of the dorsal arch base.

FIG. 10 shows the bottom side view, identifying the relationship between dorsal arch base and the clip. The separation between the clip and dorsal arch base allows the in-step-pad to slide in between while maintaining its curvatures.

DRAWINGS - - Reference Numerals

| | |
|---|---|
| 20 handle | 21 neck |
| 22 circular top | 23 dorsal arch base |
| 24 crescent indentation | 25 divider |
| 26 divider (anterior apex) | 27 divider (posterior aspect) |
| 28 butterfly top | 29 left greater wing |
| 30 right greater wing | 31 left inferior wing |
| 32 right inferior wing | 33 anterior notch |
| 34 posterior notch | 35 center body |
| 36 clip | 37 clip neck |
| 38 dorsal wrap-around DAFO | 39 dorsal wrap around supports |
| 40 Donning Facilitator | 41 in-step-pad |
| 42 bilateral grooves | |

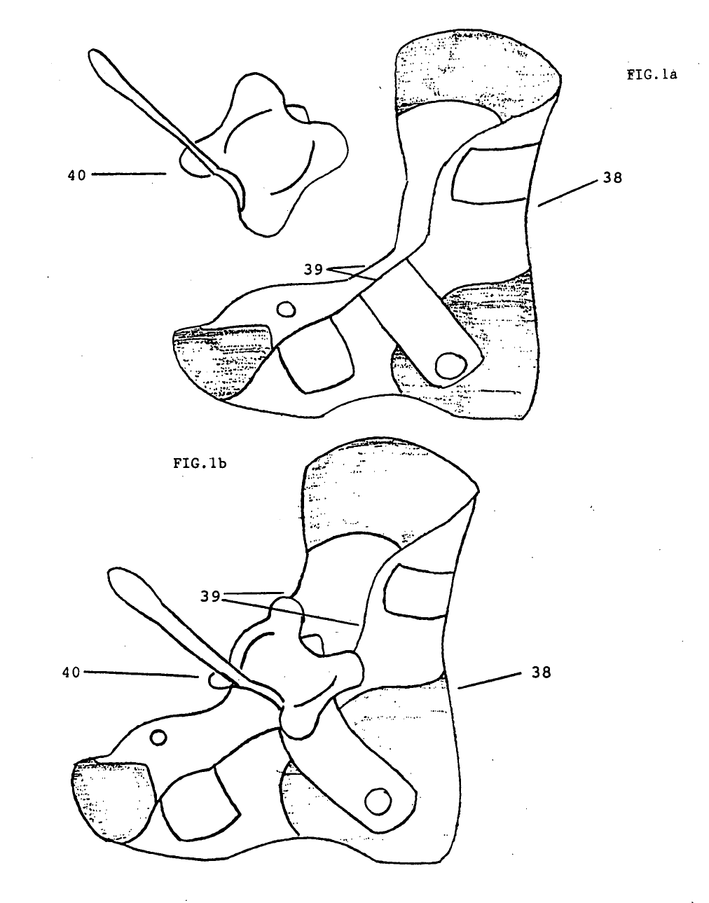

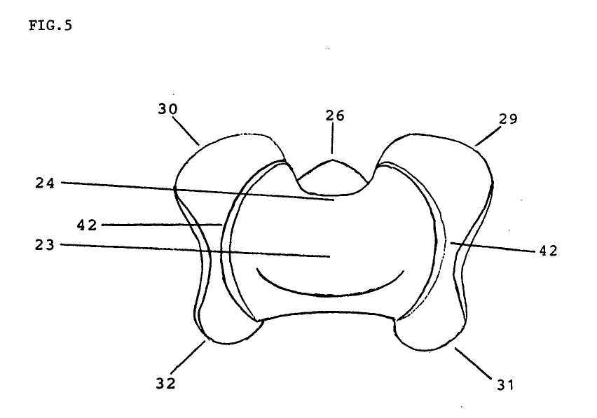

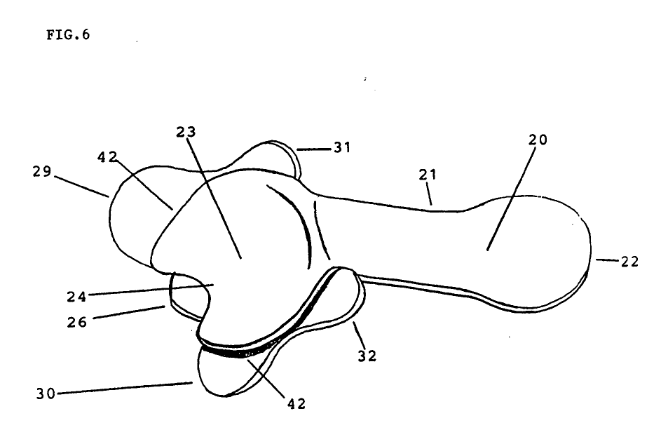

### DETAILED DESCRIPTION—FIGS. 1-7—PREFERRED EMBODIMENT

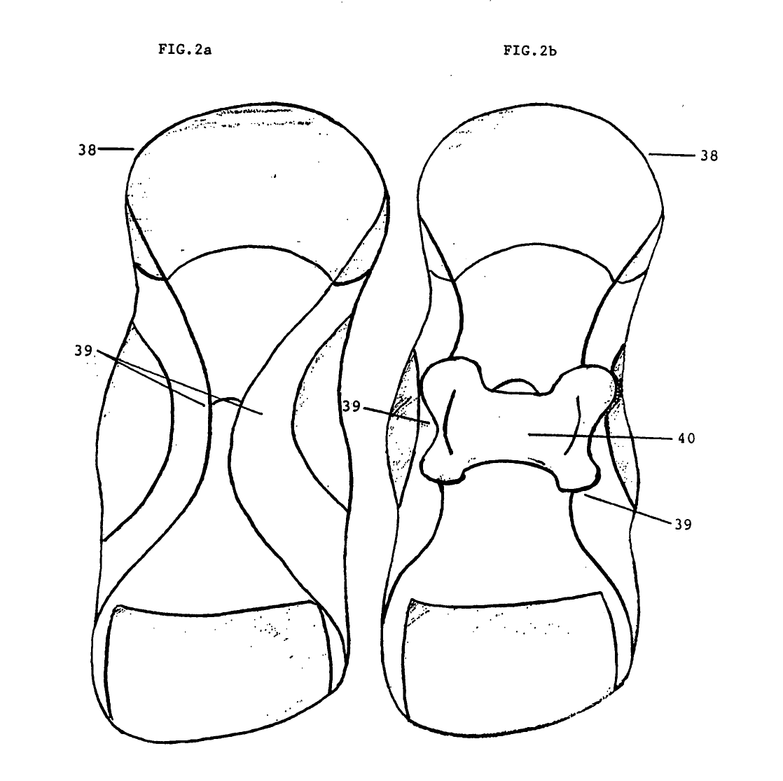

A preferred embodiment of the Donning Facilitator 40 is illustrated in FIGS. 1a-1b demonstrating the Donning Facilitator 40 and how it relates with the dorsal wrap-around DAFO 38 by showing the side view closed position and the separation between the Donning Facilitator 40 and the bilateral dorsal wrap-around supports 39 located at the anterior-dorsal aspect of the dorsal wrap-around DAFO 38. FIGS. 2a-2b shows the front view of the anterior-dorsal aspect of the dorsal wrap-around supports 39 of the dorsal wrap-around DAFO 38 and the enlarged opening caused by the separation between the Donning Facilitator 40 and the dorsal wrap-around supports 39 of the dorsal wrap-around DAFO 38.

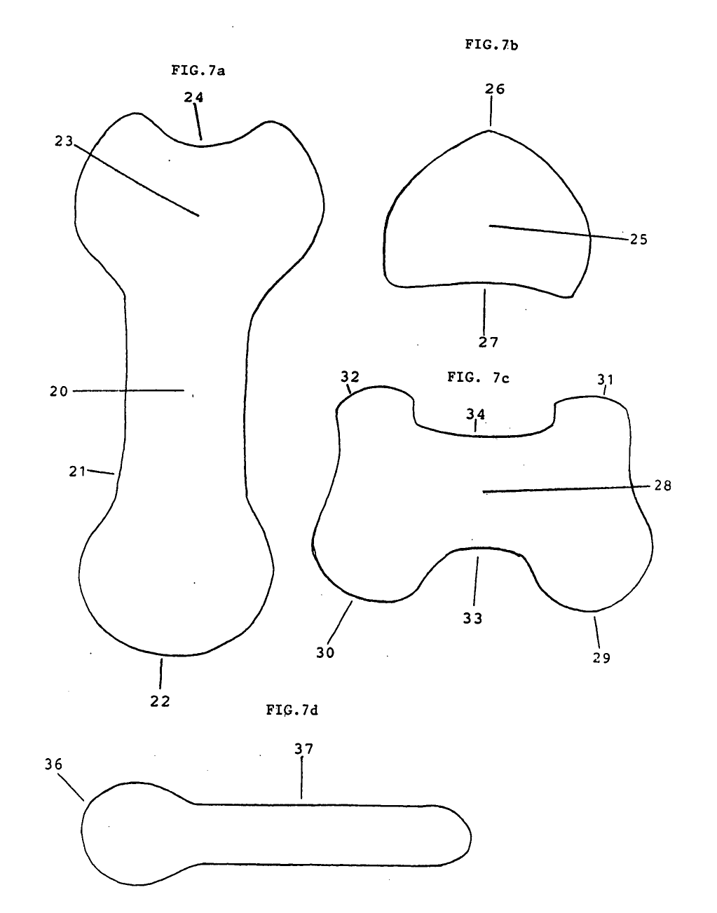

In the preferred embodiment of the Donning Facilitator 40, as illustrated in FIGS. 7a, 7b, 7c there are three separate pattern pieces that are molded to their desired shape and adhered to each other respectfully. The dorsal arch base 23 is concaved and circular in shape with a crescent indentation 24 at the anterior portion of the dorsal arch base 23. The posterior portion of the dorsal arch base 23 makes a bend transitioning into the handle 20. The divider 25 is molded into the same curvature as the dorsal arch base 23 with the anterior apex 26 slightly turned upward and then adhered to the top center portion of the dorsal arch base 23. The dividers' anterior apex 26 extends over the crescent indentation 24 of the anterior portion of the dorsal arch base 23. The butterfly top 28 is then molded to the same curvature as the divider 25 and dorsal arch base 23. Then the butterfly top 28 is adhered to the top center portion of the divider 25. The posterior notch 34 is positioned adjacent to the posterior portion of the dorsal arch base 23 where it bends into the handle 20. The anterior notch 33 of the butterfly top 28 maintains the exposed anterior apex of the divider 26. The left and right greater wings 29-30 are molded in a significant upward direction. The left and right inferior wings 31-32 are molded in a lesser upward direction. In all there are three separate pattern components that are molded to their desired shape and adhered to each other respectfully.

In the preferred embodiment, the material that makes up each component is a thermoplastic material combining malleability, strength and cohesive qualities. One such material is orthoplast, available from Johnson & Johnson Professional, Inc. of Raynham, Mass. 02767-0350 USA. However, the Donning Facilitator 40 can consist of any other material that can be molded or injection molded and then maintain the designed shape that can with stand repeated use without fracturing, such as Polymers, Poly-Olefins, or other plastic materials.

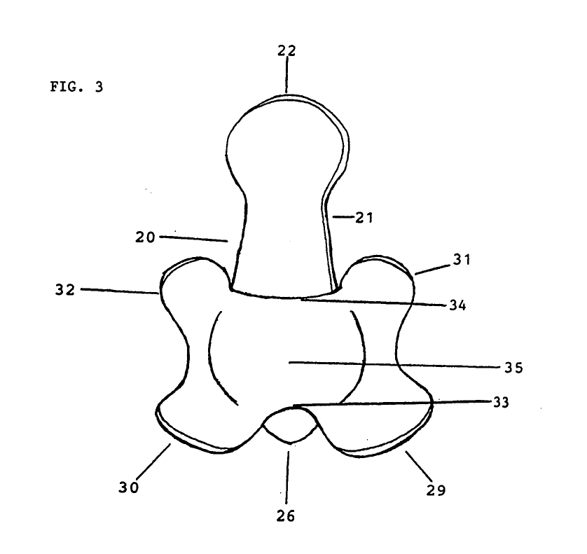

The material that makes up the Donning Facilitator 40 is typically 4 mm in thickness. As illustrated in FIG. 3 the top of the handle 20 is circular in shape named circular top 22 with a diameter roughly 4.5 cm. The divider 25 (shape similar to an arrow head) consists of the anterior apex 26 (point of reference) that gradually widens into a bilateral concaved arch shape ending at the divider's posterior aspect 27 typically 4 cm in length×4.8 cm in width. The anterior apex 26 of the divider is then slightly curved upward. This prevents the point of the anterior apex 26 from coming into contact with the dorsum of the patient's ankle.

Also illustrated in FIG. 3 the butterfly top 28 (butterfly shape) consists of six different aspects; beginning with the left greater wing 29 (diagonally placed half circle) located on the left side of the body of the butterfly top 28 diameter roughly 2 cm. The right greater wing 30 (diagonally placed half circle) located on the right side of the body of the butterfly top 28 diameter roughly 2 cm. The left inferior wing 31 (square leg) located on the posterior left side of the body of the butterfly top 28 width 2 cm×length 1 cm. The right inferior wing 32 (square leg) located on the posterior right side of the body of the butterfly top 28 width 2 cm×length 1 cm. The anterior notch 33 located front center and between the two laterally placed greater wings 29-30 The bottom of the anterior notch 33 measures approximately 2 cm in width the depth is approximately 1.5 cm. The bilateral walls of the anterior notch 33 measure approximately 1.5 cm in length each wall. The posterior notch 34 located back center and between the two laterally placed inferior wings 31-32. The bottom of the posterior notch 34 measures 3.5 cm in width. The bilateral walls of the posterior notch 34 measure approximately 8 mm in length. The curvatures of the butterfly top 28 are such that the center body 35 is curved downward while both greater wings 29-30 are curved in an upward direction. The curvatures of the inferior wings 31-32 are also curved also in an upward direction. The outer edges of each individual piece making up the Donning Facilitator 40 are beveled or rounded to avoid snagging or personal injury.

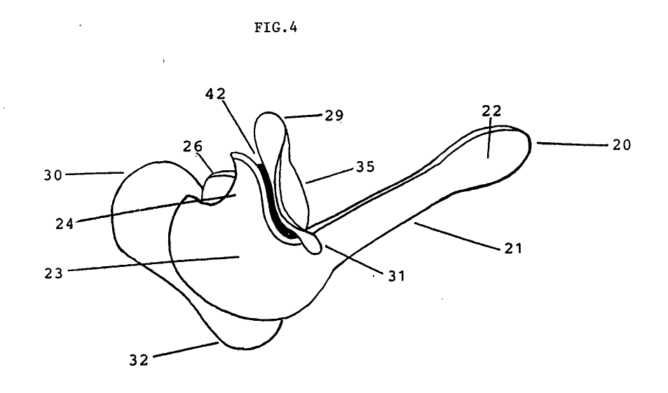

As illustrated in FIG. 4 the dorsal arch base 23 and handle 20 consist of one individual component. Starting with the dorsal arch base 23 (circular/crescent shape) the overall dimensions is roughly 6 cm in width×4 cm in depth. The handle 20 extends from the posterior portion of the dorsal arch base 23 and bends to transition into the handle 20. This typically measures 9.5 cm in length×3 cm in width at the neck 21.

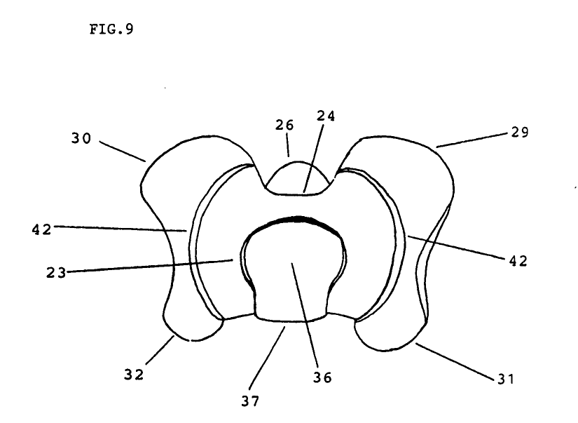

Additional Embodiments FIGS. 7-10

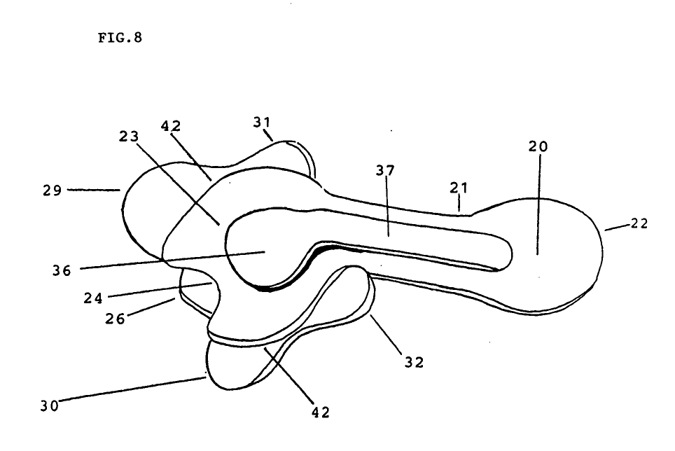

In other embodiments there can be a forth piece of material named the clip 36. As illustrated in FIG. 8 the clip neck 37 adheres to the back of the handle neck 21 and separates away prior to the bend of the posterior portion of the dorsal arch base 23. The length and width of the clip 36 and the clip neck 37 is roughly 10 cm in length×1.5 cm in width. The clip 36 is a diameter of 2 cm and is found underneath the center of the dorsal arch base 23 with approximately 4 mm of separation. The separation between the bottom of the dorsal arch base 23 and the distal end of the clip 36 is approximately 4 mm. This allows the in-step-pad 41 of the dorsal wrap-around DAFO 38 to be placed snugly in between the clip 36 and the dorsal arch base 23 prior to using the Donning Facilitator 40 to separate the dorsal wrap-around supports 39 into an enlarged opening. When the foot descends into the created enlarged opening the Donning Facilitator 40 then backs out leaving the in-step-pad 41 correctly in place.

Another embodiment of the Donning Facilitator 40 is that all the above measurements can vary proportionately in relationship to the various sizes of the dorsal wrap-around DAFO 38, which is required to fit the individual patient.

Operation—FIGS. 1-7

The Donning Facilitator 40 is a hand held adaptive device, designed for the disabled person who requires the use of the dorsal wrap-around DAFO 38. The operation of using the Donning Facilitator 40 begins with holding the Donning Facilitator 40 at the neck of the handle 21 with the apex (point of reference) of the divider 26 directed toward the bottom separation of the dorsal wrap-round supports 39. Then the dorsal wrap-around DAFO 38 is stabilized in-between the patient's opposite arm and trunk or between the patient's legs. Also if the opposite upper extremity is functional enough to stabilize and hold the dorsal wrap-around DAFO 38, then the Donning Facilitator 40 with the anterior apex divider 26 is inserted between the bottom (distal) separation of the dorsal wrap-around supports 39. At this time the dividers' 25 bilateral edges in conjunction with the bilateral grooves 42 are lined up with the bilateral edges of the dorsal wrap-around supports 39 and then the patient pushes and slides the Donning Facilitator 40 up causing the dorsal wrap around supports 39 to separate and create an enlarged opening to its widest state.

While the divider 25 is separating the dorsal wrap-around supports 39 the greater and inferior wings 29-32 are preventing the dorsal wrap-around supports 39 from sliding off or from between the bilateral grooves 42 of the Donning Facilitator 40. In addition the dorsal arch base 23 is positioned under the dorsal wrap-around supports 39 of the dorsal wrap-around DAFO 38 for additional support in the opposite direction.

Once the Donning Facilitator 40 is in the proper position between the dorsal wrap-around supports 39 of the dorsal wrap-around DAFO 38, the created enlarged opening will remain open. The patient can then reposition his hands away from the Donning Facilitator 40 and the Donning Facilitator 40 will remain in its position until the patient's foot comes down the anterior-dorsal aspect of the DAFO of the dorsal wrap-around DAFO 38. While seated or in side lying position, the patient can hold the proximal end of the dorsal wrap-around DAFO 38 with only one hand and slide the impaired foot down the created enlarged opening (anterior-dorsal aspect of the DAFO) of the dorsal wrap-around DAFO 38, similarly to sliding your foot down into an enlarged opening of a boot. At the same time as the foot is descending into the enlarged opening (anterior-dorsal aspect of the DAFO) of the dorsal wrap-around DAFO 38 the Donning Facilitator 40 then backs out and the foot slides into its intended place. The dorsal wrap-around supports 39 close down onto the dorsum of the ankle and foot.

The actual donning process only requires one hand to accomplish the task. This is the overall benefit that the Donning Facilitator 40 accomplishes, allowing a person to don their dorsal wrap around DAFO 38 with minimal effort and only one hand.

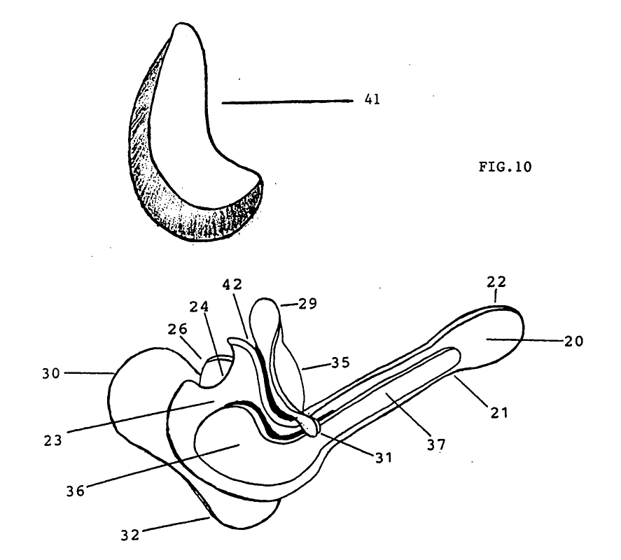

As shown in FIGS. 7d, 8,9,10 another embodiment can be a fourth piece of material named the clip 36. As illustrated in FIG. 8 the clip neck 37 adheres to the back of the handle 20 and separates away just prior to the bend of the posterior portion of the dorsal arch base 23 and continues underneath the center of the dorsal arch base 23 approximately 4 mm of separation. As shown in FIG. 10 the separation between the bottom of the dorsal arch base 23 and the distal end of the clip 36 allows the in-step-pad 41 of the dorsal wrap around DAFO 38 to be placed snugly in-between. Once the in-step-pad 41 is inserted into the Donning Facilitator 40, the same process of using the Donning Facilitator 40 is applied, although now you can insert the in-step-pad 41 within the Donning Facilitator 40. This will allow the patient to have the in-step-pad 41 placed properly at the same time as the dorsal wrap around DAFO 38 to the patient's foot and ankle.

The operation of using the Donning Facilitator 40 with the in-step-pad 41 begins by inserting the in-step-pad 41 between the clip 36 and the dorsal arch base 23. Then hold the Donning Facilitator 40 at the neck of the handle 21 with the apex (point of reference) of the divider 26 directed toward the bottom (distal) separation of the dorsal wrap-around supports 39. Then the Donning Facilitator 40 with its anterior apex divider 26 is inserted between the bottom (distal) separation of the dorsal wrap-around supports 39. At this time the dividers' 25 lateral edges in conjunction with the bilateral grooves 42 are lined up with the bilateral edges of the dorsal wrap-around supports 39 and then the patient pushes/slides the Donning Facilitator 40 up causing the dorsal wrap-around supports 39 to separate and cause an enlarged opening of its widest state.

While the divider 25 is separating the dorsal wrap-around supports 39, the greater and inferior wings 29-32 are preventing the bilateral dorsal wrap-around supports 39 from sliding off from between the bilateral grooves 42 of the Donning Facilitator 40. In addition the dorsal arch base 23 is positioned under the dorsal wrap-around supports 39.

Once the Donning Facilitator 40 is in the proper position between the dorsal wrap-around supports 39 of the dorsal wrap-around DAFO 38, the created enlarged opening will remain open. The patient can then reposition his hands away from the Donning Facilitator 40 and the Donning Facilitator 40 will remain in its position until the patient's foot comes down the created enlarged opening of the anterior-dorsal aspect of the dorsal wrap-around DAFO 38. While seated or in side-lying position, the patient can hold the proximal end of the dorsal wrap-around DAFO 38 with only one hand and slide the impaired foot down the created enlarged opening of the dorsal wrap-around DAFO 38, similarly to sliding your foot down into the enlarged opening of a boot. At the same time as the foot is descending into the created enlarged opening of the dorsal wrap-around DAFO 38 the Donning Facilitator 40 then backs out, leaving the in-step-pad 41 in place on the dorsum of the patient's foot, and the patients foot slides into its intended place.

With regards to the in-step-pad 41, the foot comes down and makes contact with the bottom of the dorsal arch base 23 and the clip 36, and the in-step-pad 41. The in-step-pad 41 with its hooked fabric attaches to the patient's sock, leaving the in-step-pad 41 correctly in place. The dorsal wrap-around supports 39 close down onto the dorsum of the ankle and foot with the in-step-pad 41 positioned between the dorsum of the foot and the underneath portion of the dorsal wrap-around supports 39. At the same time the Donning Facilitator 40 backs out from between the dorsal wrap-around supports 39.

The actual donning process requires only one functional hand to accomplish the task. The primary benefit that the Donning Facilitator 40 provides a person is to don their dorsal wrap-around DAFO 38 with minimal effort and does not require more than one hand.

Advantages:

Accordingly, several advantages of my invention are that the Donning Facilitator removes the difficulty of donning the dorsal wrap-around DAFO, providing modified independence to the patient and significant ease on the part of the caregiver:
(a) The Donning Facilitator provides the disabled person a tool to use in spreading the dorsal wrap-around supports into an enlarged opening (anterior-dorsal aspect of the DAFO) making it easy to slide the impaired foot down into its custom molded position.
(b) The temporarily enlarged state provides the disabled person the minimum amount of effort necessary to don the dorsal wrap-around DAFO on to their ankle/foot and to maximize the level of success.
(c) The improved ease of donning the dorsal wrap-around DAFO results in the improved self-confidence and gratification of the person having to use the dorsal wrap-around DAFO.
(d) To avoid the frustration commonly associated with donning the dorsal wrap-around DAFO resulting in greater compliance of wearing the dorsal wrap-around DAFO obtained by the improved ease of donning the dorsal wrap-around DAFO.
(e) To provide a disabled person away to don the dorsal wrap-around DAFO in a modified independent manner.
(f) By having the dorsal wrap-around supports spread open while not having to hold them open with both hands provides the disabled person a much easier method of donning there dorsal wrap-around DAFO by sliding their foot down into the created enlarged opening (anterior-dorsal aspect of the DAFO), similar to putting on an open boot with the use of one hand.
(g) The Donning Facilitator device will slide backwards and out as the foot descends down into the created enlarged opening (anterior-dorsal aspect of the DAFO) allowing ample space for the foot to slide into the proper position and easy exit of the Donning Facilitator.
(h) The caregiver can now don the dorsal wrap-around DAFO with the use of the Donning Facilitator and free up his or her hands from having to hold open the dorsal wrap-around supports. This then allows the caregiver ample ability to negotiate the patient ankle/foot into the dorsal wrap-around DAFO with increased ease and assurance to not startle or hurt the patient when donning the dorsal wrap-around DAFO.
(i) In addition, the hard to place in-step-pad that comes with the dorsal wrap-around DAFO can be included in the same process of donning the dorsal wrap-around DAFO by sliding the in-step-pad in between the dorsal arch base and the clip.
   Conclusion, Ramifications, and Scope:

Accordingly, the reader will see that the invention, the Donning Facilitator for Dorsal Wrap-Around Ankle Foot Orthosis provides the needed separation of the dorsal wrap-around supports of the dorsal wrap-around DAFO. The Donning Facilitator is easily and conveniently moved into the desired position between the dorsal wrap-around supports thus resulting in a created enlarged opening ideal for the disabled persons ankle/foot to slide into the custom fit position of the dorsal wrap-around DAFO. The disabled person no longer will be burdened with having to hold both sides of the dorsal wrap-around supports apart while at the same time donning the dorsal wrap-around DAFO on their ankle/foot. The bilateral grooves of the dorsal arch base of the Donning Facilitator provides significant control of the separated dorsal wrap-around supports. The Donning Facilitator is easily positioned between the dorsal wrap-around supports and then the Donning Facilitator can be let go of and the will remain stationary, allowing the disabled person the freedom to then use one hand to hold the proximal end of the dorsal wrap-around DAFO and slide their effected foot into the enlarged opening of the dorsal wrap-around DAFO. The Donning Facilitator is then backed out from between the dorsal wrap-around supports closing the dorsal wrap-around supports onto the dorsum of the disabled person's ankle/foot.

Overall the disabled person can now accomplish the task of donning the dorsal wrap-around DAFO with minimal physical effort. In addition, the caregiver will find the Donning Facilitator frees up both hands from having to hold open the dorsal wrap-around supports, resulting in ample control for the caregiver when donning the dorsal wrap-around DAFO to the patient's ankle/foot. Furthermore, the Donning Facilitator has additional advantages in that:
It can include the in-step-pad during the same process of donning the dorsal wrap-around DAFO to the persons foot/ankle. The in-step-pad can be placed within the Donning Facilitator by means of the clip. The in-step-pad will remain in position of under the dorsal wrap-around supports and on the dorsum of the ankle as the Donning Facilitator backs out.
It can include a contrasting color of the anterior apex of the divider thus allowing the patient a point of reference. Thus providing those with impaired vision increased ability to align the Donning Facilitator between the dorsal wrap around supports.
It can include a positive and negative notch between the Donning Facilitator's divider/bilateral grooves and the bilateral edges of the dorsal wrap-around supports of the dorsal wrap-around DAFO. This would allow for a lock-in system to occur between the Donning Facilitator and the dorsal wrap-around DAFO. However this could be a small indentation to simply prevent the potential sliding of the Donning Facilitator all the way through the dorsal wrap-around supports and out at the top portion of the dorsal wrap-around DAFO.
The handle of the Donning Facilitator could be built up to possibly provide a better grasp for those who have week hand strength or poor upper extremity motor control.
The Donning Facilitator can vary in size to accommodate the various sizes of dorsal wrap-around DAFO's.
The Donning Facilitator can consist of any other material that can be molded or injection molded and then maintains the designed shape that can withstand repeated use with out fracturing. This could include metals.
The clip is found underneath the center of the dorsal arch base with a slight separation, a coil type system giving a spring type response at the bend between the clip and the clip neck may also provide additional hold to the in-step-pad within the Donning Facilitator.

Although the descriptions above contain many specificities, these should not be construed as limiting the scope of the invention but as merely providing illustrations of some of the alternative embodiments of this invention.

Thus the scope of the invention should be determined by the appended claims and their legal equivalents, rather than by the examples given.

I claim:

1. For use in donning a dorsal wrap-around ankle-foot orthosis with dorsal wrap supports, a hand-held device comprising:
a) a handle; and
b) an opening mechanism
wherein said handle is attached to said opening mechanism and said opening mechanism operates by
i) separating said dorsal wrap-around supports of said dorsal wrap-around ankle-foot orthosis by sliding said opening mechanism upward through the gap between said dorsal wrap-around supports, enlarging said gap between said dorsal wrap-around supports; and
ii) holding open said dorsal wrap-around supports; and
iii) sliding out of said gap between said dorsal wrap-around supports as a foot is inserted from above into said dorsal wrap-around ankle-foot orthosis.

2. The device of claim 1 in which said handle and said opening mechanism are constructed of three distinct pieces of material and adhered together, wherein the first piece is elongated, forming said handle and forming the bottom layer of said opening mechanism; and
a) the second piece is substantially arrow-shaped, pointing in the direction said hand-held device moves while separating said dorsal wrap-around supports and adhered to the top side of the lower portion of said first piece; and
b) the third piece is larger than said second piece and adhered to the top of said second piece;
c) and wherein said second piece is sandwiched between said first piece and said third piece.

3. The device of claim 2, further comprising a fourth piece of material which is substantially spoon-shaped, in which the elongated portion of said forth piece is attached to said handle of said hand-held device, with the rounded end of said forth piece projecting below said opening mechanism, providing a means to hold an in-step pad between said rounded end of said forth piece and said opening mechanism, and allowing said in-step pad to be released as a foot is inserted from above into said dorsal wrap-around ankle-foot orthosis.

4. The device of claim 2 in which said three pieces are made of a thermoplastic material.

5. The device of claim 3 in which said three pieces and said forth piece are made of a thermoplastic material.

6. The device of claim 2 in which said three pieces are made of polymers.

7. The device of claim 3 in which said three pieces and said forth piece are made of polymers.

8. The device of claim 2 in which said three pieces are made of poly-olefins.

9. The device of claim 3 in which said three pieces and said forth piece are made of poly-olefins.

10. The device of claim 1 in which said handle and said opening mechanism are constructed together initially as a single piece, the upper portion of said single piece operating as said handle, and the lower portion of said single piece operating as said opening mechanism, wherein said opening mechanism comprises three layers and wherein the second (middle) layer is substantially arrow-shaped and pointing in the direction said hand-held device moves while separating said dorsal wrap supports; and the first and third layers (bottom and top layers) are larger in length and width than said second layer.

11. The device of claim 10, further comprising a forth layer for said opening mechanism that is below said three layers, in which said forth layer is rounded and attached to the bottom of said three layers partially along the circumference of said forth layer, forming a clip that can hold an in-step pad, and allowing said in-step pad to be released as a foot is inserted from above into said dorsal wrap-around ankle-foot orthotic.

12. The device of claim 10 in which said three pieces are made of a thermoplastic material.

13. The device of claim 11 in which said three pieces and said forth piece are made of a thermoplastic material.

14. The device of claim 10 in which said three pieces are made of polymers.

15. The device of claim 11 in which said three pieces and said forth piece are made of polymers.

16. The device of claim 10 in which said three pieces are made of poly-olefins.

17. The device of claim 11 in which said three pieces and said forth piece are made of poly-olefins.
